(19) United States
(12) Plant Patent Application Publication
(10) Pub. No.: US 2014/0345012 P1
(43) Pub. Date: Nov. 20, 2014

(54) Mandarin tree named GCM 305

(50) ;

(71) Applicant: GCM VARIEDADES VEGETALES A.I.E., Almazora (ES)

(72) Inventor: Fernando Ballester Martinavarro, Sollana (ES)

(73) Assignee: GCM VARIEDADES VEGETALES A.I.E., Almazora (ES)

(21) Appl. No.: 13/986,574

(22) Filed: May 16, 2013

(51) Int. Cl. A01H 5/00

(52) U.S. Cl. PLT/202

(57) ABSTRACT

‘GCM 305’ is a late-season maturing mandarin obtained by partial cross of the ‘KARA’ mandarin in Huelva, Spain. ‘GCM 305’ has medium-sized fruit which is essentially seedless and has good storage characteristics. The fruit is easy to peel, has a smooth rind texture, a dark orange color, a very rich sweet flavor, and a pleasant aroma.

### LATIN NAME OF THE GENUS AND SPECIES

The mandarin cultivar of this invention is botanically identified as Citrus reticulata hybrid.

### VARIETY DENOMINATION

The variety denomination is ‘GCM 305’.

### BACKGROUND OF THE INVENTION

The invention relates to a new and distinctive mandarin hybrid cultivar designated ‘GCM 305’, which was developed in Huelva, Spain.

### BRIEF SUMMARY OF THE INVENTION

‘GCM 305’ is a late-season maturing mandarin variety having medium-sized fruit which is essentially seedless and has a rich flavor.

‘GCM 305’ is distinguished from other cultivars in that it produces fruit that combine late-season maturity and medium size with an absence of neck. The fruit is easy to peel, has a smooth rind texture, a dark orange color, a very rich sweet flavor, and a pleasant aroma.

### BRIEF DESCRIPTION OF THE DRAWINGS

The figures depict various characteristics of ‘GCM 305’.

FIG. 1 shows fruit and foliage of ‘GCM 305’ taken at Huelva in February 2013.

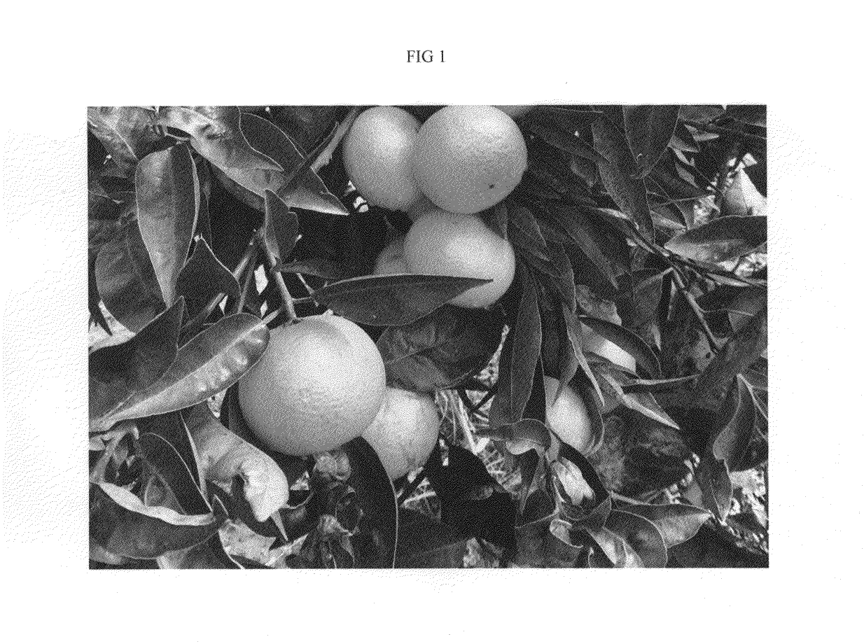

FIG. 2 shows a cross-section of ‘GCM 305’ fruit.

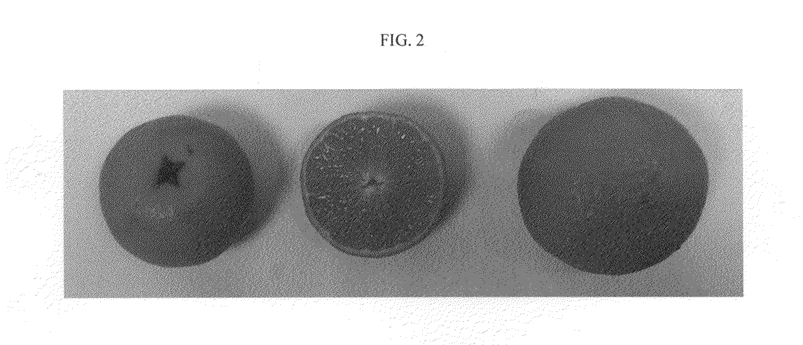

FIG. 3 shows the ‘GCM 305’ flowers.

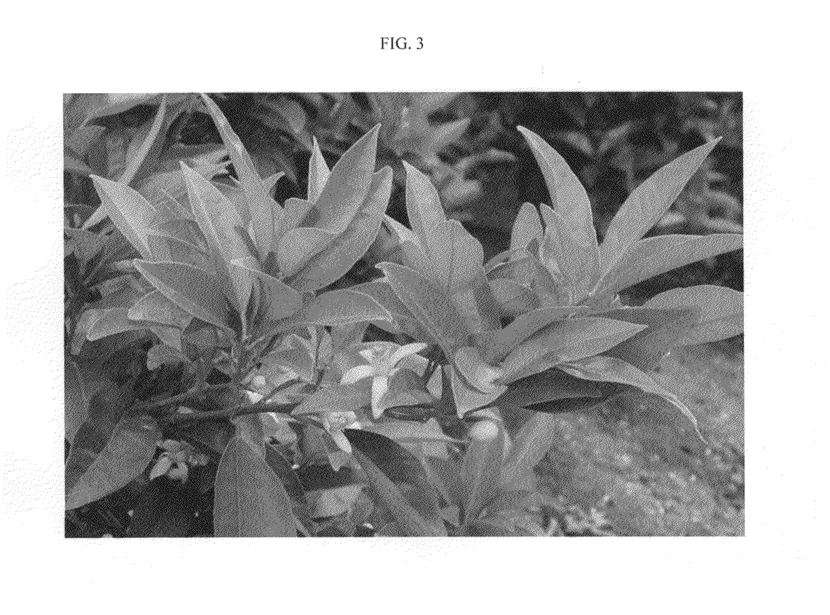

FIG. 4 shows a two-year-old ‘GCM 305’ mandarin tree grafted on a five years Carrizo citrange rootstock.

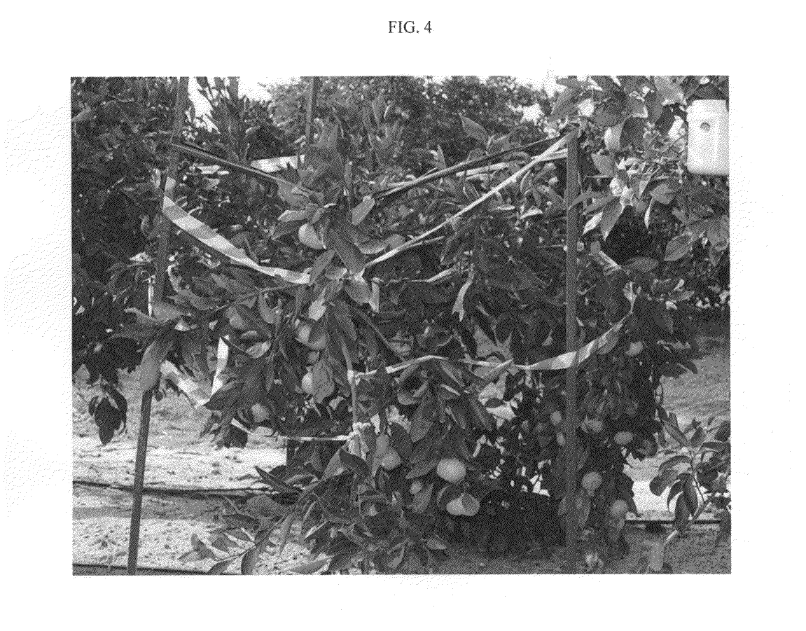

### DETAILED DESCRIPTION

‘GCM 305’ was obtained by partial cross in Huelva, Spain, of the ‘KARA’ mandarin (unpatented), which is a diploid of C. unshiu×C. nobilis (Citrus reticulata Blanco). The fruit was collected in December 2007, and seeds of the fruit were extracted and subsequently germinated during January 2008. About 350 seedlings were grown from the germinated seeds. Each seedling was grafted in March 2009 onto two-year-old Citrus macrophylla rootstocks that had been planted in 10-liter pots.

The grafted plants were cultivated and the first fruit were observed in January 2012. Observations of the fruits were made again in 2013. One of these 350 seedling was designated ‘GCM 305’.

An important characteristic of ‘GCM 305’ is that it is essentially seedless.

Studies of hand cross-pollination were made between ‘GCM 305’ and other seeded and seedless varieties. For each combination, ‘GCM 305’ was used as a female parent and as pollinator. The studies showed that self-pollinated ‘GCM 305’ is seedless, whereas the parent variety ‘KARA’ is seeded.

‘GCM 305’ is a late-maturing variety like its parent ‘KARA’, but unlike ‘KARA’ the ‘GCM 305’ variety has seedless fruit.
Tree size and growth characteristics:
   Size of tree.—Medium sized. A typical two-year-old tree is 1.80 meters high by 1.60 meters wide. A typical trunk diameter in a three-year-old rootstock is 4 cm at 9 cm above the ground.
   Shape of tree.—Spheroid, erect-drooping similar to ‘KARA’.
   Growth characteristic.—Vigorous growth.
Leaves:
   Shape.—Lanceolate.
   Petioles.—No wing on the petiole.
   Color.—Medium green (RHS 147A).
Thorns: None.
Flowers:
   Time of flowering.—Early April in Huelva, Spain.
   Type of flower.—Hermaphrodite.
   Color of open flower.—White with coloring on the lower side of petals (RHS 155C).
   Flower grouping.—Solitary in a raceme arrangement at axillary and terminal positions.
   Number of petals.—5 to 6.
   Number of anthers.—18-20.
   Color of anthers.—Light yellow (RHS 8C).
   Pollen.—Fertile. Degree of germination unknown.
Fruit:
   Time of fruit harvest.—Early February in Huelva, Spain.
   Shape.—Obloid.
   Diameter.—76 mm.
   Length.—53 mm.
   Rind.—Medium orange (RHS 30B), with medium number of oil glands. Thickness of rind at maturity is 2.5-3 mm.
   Ease of separation of rind from fruit at maturity.—Medium.
   Flesh.—Medium orange (RHS 28A), with fine texture.
   Axis at maturity.—Medium.
   Number of segments in a fruit.—9-10.
   Under self-pollination conditions.—Fruit are seedless.
   Acidity/sweetness.—1% acid content/13° Brix.
   Degree of juiciness.—51%.
   Fruit production.—Begins at 2-3 years.
   Storage characteristics.—Good for washed but unwaxed fruit. Also good for waxed fruit. No loss of rind quality or color, no loss in juice quality or deterioration in taste observed upon storage. No indication of fungal or other disease upon storage.

1. A new and distinct mandarin tree having the characteristics substantially as described and illustrated herein.
